(19) United States
(12) Patent Application Publication
(10) Pub. No.: US 2021/0322958 A1
(43) Pub. Date: Oct. 21, 2021

(54) COPPER-BASED CATALYSTS

(71) Applicant: King Abdullah University of Science and Technology, Thuwal (SA)

(72) Inventors: Kuo-Wei HUANG, Thuwal (SA); Ding-Jier YUAN, Thuwal (SA)

(21) Appl. No.: 17/273,355

(22) PCT Filed: Sep. 5, 2019
(86) PCT No.: PCT/IB2019/057498

§ 371 Date: Mar. 4, 2021

Related U.S. Application Data
(60) Provisional application No. 62/727,664, filed on Sep. 6, 2018.
(60) Provisional application No. 62/744,351, filed on Oct. 11, 2018.

(51) Int. Cl. B01J 23/78; B01J 37/03; B01J 37/02; B01J 37/18; B01J 35/02; C07C 67/40; B01J 37/08

(52) U.S. Cl. CPC B01J 23/78; B01J 37/031; B01J 37/0201; B01J 37/08; B01J 35/023; C07C 67/40; B01J 37/18

(57) ABSTRACT

Catalyst compositions comprising catalytic nanoparticles including copper distributed, dispersed on, or mixed with a promoter including magnesium oxide. Pre-catalyst compositions comprising nanoparticles including copper oxides or copper hydroxide distributed, dispersed on, or mixed with a promoter including magnesium oxide. The catalysts are used in a method of producing at least methyl formate and hydrogen by non-oxidative dehydrogenation of methanol, optionally comprising reducing a pre-catalyst in hydrogen at a select temperature to obtain a catalyst comprising catalytic nanoparticles including copper distributed, dispersed on, or mixed with a promoter including magnesium oxide, flowing a fluid composition containing at least methanol over the catalyst to produce methyl formate and hydrogen, and recovering one or more of the methyl formate and hydrogen. A method of preparing catalyst compositions is disclosed. Alternatively or in addition to copper, the catalytic metal can be palladium, nickel or platinum. Alternatively or in addition to magnesium oxide the promoter can comprise zinc oxide, zirconium oxide, silica, calcium oxide, strontium oxide, barium oxide, lanthanum III oxide, gallium oxide, alumina, cerium oxide, vanadium oxide, chromium oxide, titanium oxide, tin oxide, and combinations or mixtures thereof.

### BACKGROUND

Methyl formate (MF) is an essential reactive intermediate in C1 chemistry. It is widely used for the synthesis of numerous value-added products in the chemical industry, including ethylene glycol, N, N dimethyl formamide (DMF), methyl glycolate, acetic acid, methyl propionate, and formamide. MF is also a highly valuable chemical that can directly be used as antiseptic, solvent, and gasoline additive. Several modes of synthesis of MF have been reported in the literature. They involve the selective oxidation of methanol, dehydrogenation of methanol, hydrogenation of CO₂ to MF, dimerization of formaldehyde, carbonylation of methanol and esterification of methanol with formic acid, with the first two synthetic routes being the most popular for the commercial production. In comparison to the selective oxidation reaction, the non-oxidative dehydrogenation reaction not only avoids the addition of an oxidant, meaning that it is not only a safer reaction to lower the production costs, but it also produces hydrogen as a value-added by-product that can be used as a fuel or reducing agent in various industries.

The direct dehydrogenation reaction of methanol to produce MF is described in details in Scheme 1. While several homogeneous systems have been developed for dehydrogenative homocoupling of primary alcohols to the corresponding esters, the formyl group of MF from methanol tends to undergo further dehydrogenation reactions. On the other hand, for heterogeneous systems, the products obtained through dehydrogenation of methanol can be diverse, and many factors can influence the catalytic performance. For example, an acidic support will lead to an increasing products resulting from a dehydration process (e.g. dimethyl ether (DME)), and higher temperatures favor to form gaseous product such as CO or CO₂. While copper can be used for MF synthesis, there are other materials that can act as catalysts for this reaction, including palladium, nickel, and platinum. However, despite the high number of studies on the non-oxidative dehydrogenation of methanol to form MF, the formation rates of MF obtained thus far are not sufficiently high for practical use, and a catalyst with a practically long lifetime still needs to be discovered.

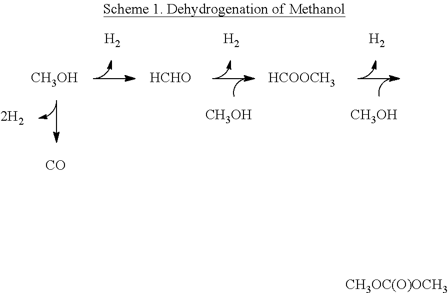

### SUMMARY

In general, embodiments of the present disclosure describe catalyst compositions, methods of preparing catalyst compositions, and methods of producing at least methyl formate by non-oxidative dehydrogenation of methanol.

Embodiments of the present disclosure describe a catalyst composition comprising catalytic nanoparticles distributed, dispersed on, or mixed with a promoter. In an embodiment, the catalyst composition comprises catalytic nanoparticles distributed on a surface of a promoter, wherein the catalyst nanoparticles include Cu nanoparticles and the promoter includes MgO. In an embodiment, the Cu nanoparticles can include one or more of CuO nanoparticles, Cu₂O nanoparticles, and Cu⁰ nanoparticles.

Embodiments of the present disclosure further describe a method of preparing a catalyst composition comprising one or more of the following steps: dissolving a metal precursor and a promoter precursor in an aqueous solution to form a precursor solution; adding, optionally under stirring, a precipitating agent to the precursor solution to form a co-precipitate; calcinating the co-precipitate at a first select temperature to form a pre-catalyst; impregnating the pre-catalyst with a Pd precursor to obtain an impregnated pre-catalyst; calcinating the impregnated pre-catalyst at a second select temperature; and reducing the pre-catalyst or impregnated pre-catalyst in a reducing atmosphere at a third select temperature to obtain a catalyst composition including catalytic nanoparticles and a promoter.

Embodiments of the present disclosure further describe a method of producing at least methyl formate by non-oxidative dehydrogenation of methanol comprising one or more of the following steps: reducing a pre-catalyst in hydrogen at a select temperature to obtain a catalyst, flowing a fluid composition containing at least methanol over the catalyst to produce methyl formate and hydrogen, and recovering one or more of the methyl formate and hydrogen.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

Reference is made to illustrative embodiments that are depicted in the figures, in which:

FIG. 1 is a flowchart of a method of preparing a pre-catalyst and/or catalyst, according to one or more embodiments of the present disclosure.

FIG. 2 is a flowchart of a method of non-oxidative dehydrogenation of methanol, according to one or more embodiments of the present disclosure.

FIGS. 3A-3B are XRD patterns for: a) fresh catalysts, b) spent catalysts, according to one or more embodiments of the present disclosure.

FIGS. 4A-4J are TEM images for: a) Cu₃MgO₇-fresh; b) Cu₃MgO₇-spent; c) 1Pd/Cu₃MgO₇-fresh; d) 1Pd/Cu₃MgO₇-spent; e) Cu₅MgO₅-fresh; f) Cu₅MgO₅-spent; g) 1Pd/Cu₅MgO₅-fresh; h) 1Pd/Cu₅MgO₅-spent; i) Cu₇MgO₃-fresh; j) Cu₇MgO₃-spent, according to one or more embodiments of the present disclosure.

FIG. 5 is a graphical view of Cu-LMM XPS study of catalysts, according to one or more embodiments of the present disclosure.

FIG. 6 is a graphical view of H₂-TPR study of catalysts, according to one or more embodiments of the present disclosure.

FIG. 7 is a graphical view of CO₂-TPD study of catalysts, according to one or more embodiments of the present disclosure.

FIGS. 8A-8B are graphical views showing the effect of Cu/MgO ratio on: a) catalytic reaction and b) deterioration rate, according to one or more embodiments of the present disclosure.

FIGS. 9A-9C are graphical views showing the effect of palladium (Pd) on the catalytic reaction: a) methanol conversion, b) methyl formate selectivity, c) formation rate of methyl formate (*Reaction condition: T=250° C., carrier gas flow (N₂)=50 mL/min, feedstock (liquid methanol)=0.1 mL/min, catalyst=100 mg, WHSV_{MeOH}=47.4 h⁻¹, 30 h), according to one or more embodiments of the present disclosure.

FIG. 10 is a graphical view showing long-term catalytic test (Reaction condition: T=250° C., carrier gas flow (N₂)=50 mL/min, feedstock (liquid methanol)=0.1 mL/min, catalyst=100 mg, WHSV_{MeOH}=47.4 h⁻¹, 100 h), according to one or more embodiments of the present disclosure.

FIG. 11 is a graphical view showing reuse test of catalytic reaction (Reaction condition: T=250° C., carrier gas flow (N₂)=50 mL/min, feedstock (liquid methanol)=0.1 mL/min, catalyst (Cu₅MgO₅-CP)=100 mg, WHSV_{MeOH}=47.4 h⁻¹, 50 h for each cycle), according to one or more embodiments of the present disclosure.

FIG. 12 is a graphical view showing thermogravimetric analysis of spent Cu₅MgO₅-CP (*Coke weight=Final W.L.−W.L. during oxidation−W.L after water remove (under 100° C.)), according to one or more embodiments of the present disclosure.

### DETAILED DESCRIPTION

The present disclosure relates to catalyst compositions for use in the synthesis of methyl formate by the non-oxidative dehydrogenation of methanol. The catalyst compositions of the present disclosure introduce a basic promoter, such as MgO, as a source of basic sites to enhance the conversion of methanol and rate of formation of methyl formate. The basic promoter can further serve as a catalyst support for the active sites of the catalyst. For example, catalytic nanoparticles, such as metallic Cu nanoparticles, can be distributed or dispersed on, or in some cases mixed with, the promoter. In some embodiments, the catalyst compositions can further be impregnated with a dopant, such as Pd. The presence of the dopant can be used to achieve a specific activity for methyl formate synthesis, as well as reduce coking and prolong the lifetime of the catalyst. Each of these components can be tuned to provide catalyst compositions that are not only highly stable and reusable, but also superior to conventional catalysts with respect to, among other things, MF formation rate, MF selectivity, and methanol conversion.

The present disclosure further relates to novel methods of preparing the catalyst compositions. In particular, the catalyst compositions can be prepared according to a novel co-precipitation method. The methods described herein can impart characteristics to the catalyst that are favorable for the conversion of methanol to methyl formate. For example, the co-precipitation method can increase the number of active sites on the surface of the promoter, without observing significant aggregation of the catalytic nanoparticles, which would result in fewer active sites. The method of preparing the catalyst compositions can proceed by, for example, adding a precipitating agent to an aqueous solution containing a metal precursor and a promoter precursor. The co-precipitate formed can be subjected to calcination to form a pre-catalyst and then activated in, for example, H₂, for the non-oxidative dehydrogenation of methanol.

The present disclosure further relates to methods of using the catalyst compositions to produce methyl formate and hydrogen by non-oxidative dehydrogenation of methanol. Unlike conventional methods and catalyst compositions, the methods of the present disclosure can proceed without the use of any oxidant and can produce hydrogen as a value-added by-product. The catalyst compositions described herein achieve high MF formation rates and high MF selectivity. The catalyst compositions exhibit excellent thermal stability over long reaction periods. Following a reaction cycle, the catalyst compositions can be easily regenerated by calcination and activated for reuse in one or more reaction cycles. During reuse, the catalyst compositions can exhibit a catalytic performance that is the same or at least similar to its performance in the first cycle.

### Definitions

The terms recited below have been defined as described below. All other terms and phrases in this disclosure shall be construed according to their ordinary meaning as understood by one of skill in the art.

As used herein, “catalyst composition(s)” refers to catalysts and pre-catalysts. A “catalyst” generally refers to a material that is catalytically active in a reaction. A “pre-catalyst” generally refers to a material that is catalytically inactive in a reaction and that is capable of becoming catalytically active (e.g., capable of becoming a catalyst) upon being activated. In one example, a “pre-catalyst” may be activated by exposure to a reducing atmosphere, such as H₂, to transform the pre-catalyst to a catalyst that is catalytically active in a reaction.

As used herein, “catalytic nanoparticle(s)” refers to nanoparticles capable of catalyzing a reaction. The term “catalytic nanoparticles” is to be understood and appreciated to include nanoparticles that are present in an active state and thus capable of catalyzing a reaction, as well as nanoparticles that are present in an inactive state and that need to be activated before being capable of catalyzing a reaction. For example, “catalytic nanoparticles” may include catalytically inactive copper oxide nanoparticles, such as CuO and/or Cu₂O, formed by oxidation of Cu via calcination, as well as catalytically active metallic copper nanoparticles, such as Cu⁰, formed by reduction of CuO and/or Cu₂O in, for example, H₂.

As used herein, “promoter” refers to a species that can be added to a catalyst to improve one or more properties of the catalyst.

As used herein, “dissolving” refers to a form of contacting in which two or more components are brought into physical contact, or immediate or close proximity. It can include dissolution of a first component (e.g., a solute) in a second component (e.g., a solvent).

As used herein, “adding” refers to a form of contacting in which two or more components are brought into physical contact, or immediate or close proximity.

As used herein, “calcining” or “calcinating” refers to heating to or at a temperature.

As used herein, “reducing” refers to exposing or subjecting to a reducing environment or atmosphere.

As used herein, “flowing” refers to a form of contacting in which two or more components are brought into physical contact, or immediate or close proximity Examples of flowing can include, but are not limited to, feeding, passing, introducing, injecting, and pumping.

As used herein, “recovering” refers to obtaining any chemical species or component that was present in, participated in, and/or produced by a chemical reaction.

Embodiments of the present disclosure describe catalyst compositions for non-oxidative dehydrogenation of methanol to produce methyl formate and hydrogen. The term “catalyst compositions” can refer to catalysts and pre-catalysts. The catalyst compositions generally comprise catalytic nanoparticles and a promoter. The catalytic nanoparticles can be distributed, dispersed on, or mixed with, the promoter. For example, the catalytic nanoparticles can be about uniformly distributed or dispersed on a surface of a promoter. In addition or in the alternative, the catalytic nanoparticles and the promoter can form a mixture, such as a homogenous mixture. A skilled person will readily appreciate that other configurations can be achieved without departing from the invention of the present disclosure.

The catalytic nanoparticles can serve as or be capable of serving as the active sites on the catalyst that catalyze the non-oxidative dehydrogenation reaction. In an embodiment, the catalytic nanoparticles include Cu nanoparticles. For example, the catalytic nanoparticles can include copper in elemental or metallic form, such as Cu⁰ nanoparticles or metallic Cu nanoparticles, or they can include copper compounds that are capable of being reduced to elemental or metallic copper. Suitable copper compounds can include, but are not limited to, copper oxides, copper hydroxides, or combinations thereof. For example, the copper compounds can include one or more of copper (I) oxide (Cu₂O), copper (II) oxide (CuO), copper (III) oxide (Cu₂O₃), copper peroxide (CuO₂), and cupric hydroxide (Cu(OH)₂). In an embodiment, the catalytic nanoparticles include CuO nanoparticles, Cu₂O nanoparticles, and combinations or mixtures thereof. In addition or in the alternative, the catalytic nanoparticles can include a metal, or a metal compound including a metal, wherein the metal is selected form the group consisting of palladium (Pd), nickel (Ni), platinum (Pt), and combinations or mixtures thereof.

The promoter can serve as a source of basic sites that enhance the performance of the catalyst (e.g., that enhance the formation rate of methyl formate, among other things). The promoter can optionally further serve as a catalyst support for the catalytic nanoparticles. The promoter generally includes a metal oxide that is chemically and/or physically stable at high temperatures, such as refractory metal oxides. An example of a suitable promoter is magnesium oxide (MgO), which can be provided as a MgO cluster and/or as MgO flakes. The MgO promoter can provide a source of basic sites. The strength of the basic sites can range from, for example, any one or more of no basic sites to weak basic sites to medium-strong basic sites to strong basic sites, among others. In addition or in the alternative, the promoter can include other metal oxides selected from the group consisting of magnesium oxide (MgO), zinc oxide (ZnO), zirconium oxide (ZrO₂), silica (SiO₂), (SiO), calcium oxide (CaO), strontium oxide (SrO), barium oxide (BaO), lanthanum III oxide (La₂O₃), gallium oxide (Ga₂O₃), alumina (Al₂O₃), cerium oxide (CeO₂), vanadium oxide (V₂O₅), chromium oxide (Cr₂O₃), titanium oxide (TiO₂), tin oxide (SnO₂), and combinations or mixtures thereof.

In one embodiment, the catalyst compositions can include Cu nanoparticles and MgO. In these embodiments, the catalyst compositions can be represented by chemical formula (I):

CuₓMgO_{y} (I)

wherein x/y is a weight ratio of Cu/MgO. The weight ratio is typically based on the amount of Cu and MgO present in the final catalyst composition, but it can be also based on the amount of Cu and MgO present in the precursor components used to prepare the catalyst compositions. The weight ratio of Cu/MgO can range from about 1/10 to about 10/1. Examples of catalyst compositions represented by the chemical formula (I) can include one or more of the following: Cu₁MgO₉, Cu₂MgO₈, Cu₃MgO₇, Cu₅MgO₅, and Cu₇MgO₃. In an embodiment, the catalyst composition is Cu₅MgO₅. In an embodiment, the catalyst compositions can include CuO, Cu, and/or MgO in a crystal phase.

The catalyst compositions can optionally further comprise an impregnated species, or a dopant, such as palladium (Pd). The addition of Pd can be used to further enhance the performance of the catalyst. In an embodiment, the catalyst compositions include a homogenous dispersion of the dopant (e.g., Pd).

In one embodiment, the catalyst compositions can include Cu nanoparticles, MgO, and Pd. In these embodiments, the catalyst compositions can be represented by chemical formula (II):

zPd/CuₓMgO_{y} (II)

wherein z is a weight % of Pd and x/y is a weight ratio of Cu/MgO, as described above. The weight % of Pd can be based on either the amount of Pd present in the precursor components used to prepare the catalyst compositions or it can be based on the amount of Pd present in the final catalyst composition. The weight % of Pd, z, can range from about 0.01 wt % to about 99 wt %. In an embodiment, the value of z is about 1. For example, catalyst compositions represented by the chemical formula (II) can include 1Pd/CuₓMgO_{y}, wherein CuₓMgO_{y} is represented by one or more of the following chemical formulas: Cu₁MgO₉, Cu₂MgO₈, Cu₃MgO₇, Cu₅MgO₅, and Cu₇MgO₃.

A ratio of Cu/MgO, and optionally the weight % of Pd, can be tuned or balanced to achieve a desired catalytic performance. For example, a ratio of catalytic nanoparticles to promoter, and optionally the weight % of Pd, can be varied to adjust a morphology and textural properties of the catalyst compositions, as well as the strength and/or presence of basic sites from the promoter. In an embodiment, the Cu loading can be adjusted to favor the formation of smaller Cu nanoparticles which provide more active sites, rather than larger Cu clusters which provide comparatively fewer active sites. In an embodiment, the MgO content can be adjusted to favor the formation of fewer medium-strong basic sites to decrease a relative rate and amount of coke formation. In an embodiment, the Pd content can be adjusted to increase catalyst lifetime, as well as enhance the reduction ability of Cu oxides. In this way, a desired methanol conversion, MF selectivity, MF formation rate, and catalyst lifetime can be achieved by tuning or adjusting one or more of the Cu content, MgO content, and Pd content.

A BET surface area of the catalyst compositions can range from about 20.4 m²/g to about 67.4 m²/g. A pore volume of the catalyst compositions can range from about 0.54 cm³/g to about 0.75 cm³/g. An average pore diameter of the catalyst compositions can range from about 100 nm to about 400 nm. An average particle size of the catalytic nanoparticles can range from about 5 nm to about 25 nm.

In an embodiment, the catalyst compositions can include one or more of Cu₁MgO₉, Cu₂MgO₈, Cu₃MgO₇, 1Pd/Cu₃MgO₇, Cu₅MgO₅, 1Pd/Cu₅MgO₅, Cu₇MgO₃, Ni/ZnO, Ni/SiO₂, Ni/ZrO₂, Pd/ZnO, Pd/SiO, Pd/ZrO₂, Pt/ZnO, Pt/SiO₂, and Pt/ZrO₂.

Embodiments of the present disclosure further describe methods of preparing the catalyst compositions described herein. For example, the catalyst compositions can be prepared by co-precipitation methods, incipient wetness impregnation methods, or combinations thereof. The catalyst compositions prepared according to the methods of the present disclosure can be activated for non-oxidative dehydrogenation of methanol by reducing the pre-catalysts in hydrogen, carbon monoxide, or carbon. The activated pre-catalysts or catalysts can then be used to produce methyl formate and hydrogen by the non-oxidative dehydrogenation of methanol.

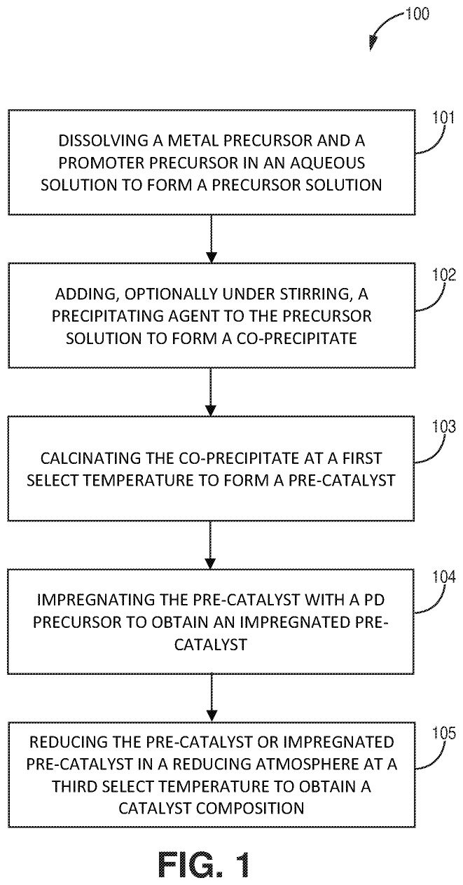

FIG. 1 is a flowchart of a method of preparing catalyst compositions, according to one or more embodiments of the present disclosure. As shown in FIG. 1, the method 100 comprises one or more of the following steps: dissolving 101 a metal precursor and a promoter precursor in an aqueous solution to form a precursor solution; adding 102, optionally under stirring, a precipitating agent to the precursor solution to form a co-precipitate; calcinating 103 the co-precipitate at a first select temperature to form a pre-catalyst; impregnating 104 the pre-catalyst with a Pd precursor to obtain an impregnated pre-catalyst; and reducing 105 the pre-catalyst or impregnated pre-catalyst in a reducing atmosphere at a third select temperature to obtain a catalyst composition including catalytic nanoparticles and a promoter.

The step 101 includes dissolving a metal precursor and a promoter precursor in an aqueous solution to form a precursor solution. The metal precursor and promoter precursor generally include water-soluble salts of the metal and promoter species, respectively. For example, the metal precursor and promoter precursor can include water-soluble salts in the form of nitrates and halides. This shall not be limiting, however, as a skilled person will readily appreciate that other precursor components can be used herein without departing from the invention of the present disclosure. In an embodiment, the metal precursor is a Cu precursor. Examples of suitable Cu precursors include, but are not limited to, Cu nitrates and/or Cu halides. In an embodiment, the Cu precursor includes one or more of Cu(NO₃)₂.3H₂O, CuSO₄.3H₂O, CuCl₂, CuBr₂, and Cu(OAc)₂.3H₂O. In an embodiment, the promoter precursor is a Mg precursor. Examples of suitable Mg precursors include, but are not limited to Mg nitrates and/or Mg halides. In an embodiment, the promoter precursor is Mg(NO₃)₂.6H₂O. In these embodiments, a weight ratio of the Cu precursor to the Mg precursor can range from about 1/10 to about 10/1. In addition or in the alternative, the metal precursor can include a water-soluble salt of a metal selected from the group consisting of Pd, Ni, and Pt. The promoter precursor can include a water-soluble salt of a promoter species selected from the group consisting of Zn, Zr, Si, Ca, Sr, Ba, La, Ga, Al, Ce, V, Cr, Ti, and Sn.

The step 102 includes adding a precipitating agent to the precursor solution to form a co-precipitate. One of the precipitating agent and precursor solution can be added dropwise to the other. For example, the precipitating agent can be added dropwise to the precursor solution, optionally under stirring. The amount of the precipitating agent added, or the rate at which the precipitating agent is added, can be controlled to maintain a suitable pH of the solution, such as a pH of about 8 to a pH of about 10. The precipitating agent can include any suitable precipitating agent. Examples of suitable precipitating agents include, but are not limited to, one or more of K₂CO₃, NH₄OH, NaOH, (NH₄)₂CO₃, and Na₂CO₃. Upon the addition of the precipitating agent to the precursor solution, or the precursor solution to the precipitating agent, either of which can optionally proceed under stirring, the co-precipitate can be formed.

The step 103 includes calcinating the co-precipitate at or to a first select temperature to form a pre-catalyst. The first select temperature can range from about 200° C. to about 500° C. For example, in an embodiment, the first select temperature is about 400° C. The co-precipitate can be calcined at the first select temperature sufficient to obtain a pre-catalyst that includes one or more metal oxides, or a mixture of one or more metal oxides. The metal oxides of the pre-catalyst can include reducible metal oxides and refractory metal oxides. For example, in an embodiment, the pre-catalyst can include a mixture of metal oxides of Cu and Mg, such as one or more of CuO, Cu₂O, and MgO. The CuO and Cu₂O, each of which can be present as copper oxide nanoparticles, can be considered reducible metal oxides; whereas MgO can be considered a refractory metal oxide that is physically and chemically stable at high temperatures (e.g., that is not reduced). The resulting pre-catalyst can include any of the pre-catalysts described herein.

The step 104 is optional and includes impregnating the pre-catalyst with a Pd precursor to obtain an impregnated pre-catalyst. This step can be performed where it is desired to introduce Pd into the catalyst composition. The Pd can be introduced by impregnation methods, such as incipient wetness impregnation. For example, the pre-catalyst can be impregnated with Pd by contacting the pre-catalyst with an aqueous solution of a Pd precursor, such as Pd(NO₃)₂.2H₂O. The contacting should be sufficient to impregnate the pre-catalyst with the solution of Pd precursor and can proceed by dropwise addition, among other techniques. Upon contacting, the pre-catalyst can be dried or heated for a duration sufficient to remove a suitable amount of solvent and deposit Pd on the pre-catalyst. The pre-catalyst can then be calcined at a select temperature (e.g., about 300° C.) for a suitable duration (e.g., about 4 h). The resulting impregnated pre-catalyst can include any of the pre-catalysts described herein.

The step 105 is optional and includes reducing the pre-catalyst in H₂ at a second select temperature. In this step, the pre-catalysts can be loaded into a reaction vessel, such as a fixed-bed reactor, among other types, and reduced prior to, or during, the non-oxidative dehydrogenation of methanol reaction. In the reaction vessel, the pre-catalyst can be subjected or exposed to a reducing environment or atmosphere sufficient for the reducible metal oxides of the pre-catalyst to be reduced to, for example, metallic or elemental form through the loss of oxygen. For example, in an embodiment, the copper oxide nanoparticles of the pre-catalysts are reduced to metallic Cu nanoparticles, and the MgO is substantially irreducible. The pre-catalysts can be reduced in hydrogen or carbon monoxide, preferably hydrogen or dilute hydrogen, which can include an inert species, such as nitrogen or argon, among others. The second select temperature can range from about 200° C. to about 300° C. In an embodiment, the second select temperature is about 250° C. The reducing should proceed for a sufficient duration, such as about 3 h. The resulting catalyst composition can include any of the catalyst compositions described herein.

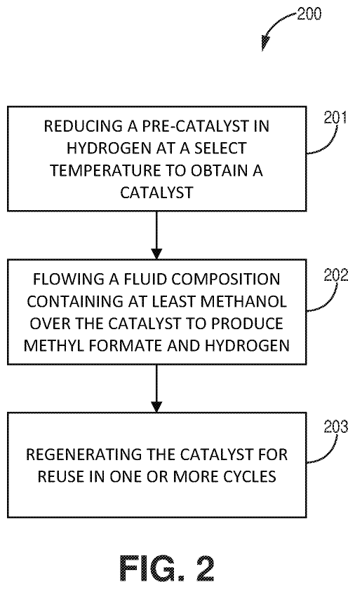

FIG. 2 is a flowchart of a method of producing at least methyl formate by non-oxidative dehydrogenation of methanol, according to one or more embodiments of the present disclosure. As shown in FIG. 2, the method 200 can comprise one or more of the following steps: reducing 201 a pre-catalyst in hydrogen at a select temperature to obtain a catalyst; flowing 202 a fluid composition containing at least methanol over the catalyst to produce methyl formate and hydrogen; and regenerating 203 the catalyst for reuse in one or more reaction cycles.

The step 201 includes reducing a pre-catalyst in hydrogen at a select temperature to obtain a catalyst. The pre-catalyst can be reduced according to any of the methods described in the present disclosure. For example, the pre-catalyst can be reduced in a fixed-bed reactor using, for example, dilute hydrogen (e.g., hydrogen combined with an inert or non-reactive species, such as N₂) for about 3 h. The pre-catalyst can include any of the pre-catalysts of the present disclosure. For example, the pre-catalyst can include a mixture of CuO, Cu₂O, and MgO. The catalyst can include any of the catalysts of the present disclosure. For example, the catalyst can include copper nanoparticles and MgO, optionally with impregnated Pd, wherein the copper nanoparticles are metallic Cu nanoparticles. In an embodiment, the catalyst compositions can be represented by any one of the chemical formulas (I) and/or (II), as described above.

The step 202 includes flowing a fluid composition containing at least methanol over a catalyst to produce methyl formate and hydrogen. Examples of flowing include, but are not limited to, one or more of flowing, feeding, and passing. The flowing can proceed at a temperature ranging from about 200° C. to about 350° C., such as about 250° C. The fluid composition can further be provided from any source, either industrial or commercial sources, or non-industrial or non-commercial sources. In an embodiment, the fluid composition can include one or more of methanol, ethanol, propanol, and butanol, among other chemical species. The fluid composition can be provided in any phase (e.g., gas, vapor, liquid, etc.). For example, in an embodiment, the fluid composition can include a liquid methanol feed stock that is vaporized in an evaporator and then fed to a reactor after the methanol vapor is combined with a carrier gas, such as N₂.

The dehydrogenation of methanol can proceed without the use of any oxidant. The catalyst can exhibit a MF selectivity of at least about 80%, with selectivities towards CO and CO₂, among other undesirable species, as low as about 1%. The catalysts can achieve a methanol conversion of at least about 10%, at least about 11%, at least about 12%, at least about 13%, at least about 14%, at least about 15%, or at least about 16%. The catalysts can also achieve a MF formation rate of at least about 10 g_{MF}/g_{cat}·h, at least about 11 g_{MF}/g_{cat}·h, at least about 12 g_{MF}/g_{cat}·h, or at least about 13 g_{MF}/g_{cat}·h. The catalysts can maintain, for example, at least about 80% of the original activity over the course of a reaction time of about 100 h, achieving high methanol conversion (e.g., at least about 79% conversion) and high MF formation rates (e.g., at least about 80% of original MF formation rate). Upon flowing methanol over the catalyst, one or more of the reaction products including methyl formate and hydrogen can optionally be separated from one or more other chemical species and recovered.

In an embodiment, a methyl formate formation rate can range from about 1.3 g_{MF}/g_{cat}·h to about 13.1 g_{MF}/g_{cat}·h. In an embodiment, a methanol conversion can range from about 1.6% to about 16.7%. In an embodiment, a methyl formate selectivity can range from about 88.1% to about 93.8%.

The step 203 includes regenerating the catalyst for reuse in one or more reaction cycles. A spent catalyst can be readily regenerated and reduced for reuse in additional reaction cycles (e.g., at least 4 or more cycles) by calcinating the spent catalyst at a temperature of about 400° C. for about 3 h, under air atmosphere, sufficient to remove deposits, such as coking, among others, and reducing the catalyst as previously described herein. Upon regenerating the catalyst, it can be reused in one or more cycles to produce additional methyl formate and hydrogen by non-oxidative dehydrogenation of methanol. Such catalysts can exhibit, among other things, at least 80% conversion and constant MF selectivity.

The following Examples are intended to illustrate the above invention and should not be construed as to narrow its scope. One skilled in the art will readily recognize that the Examiners suggest many other ways in which the invention could be practiced. It should be understand that numerous variations and modifications may be made while remaining within the scope of the invention.

### Example 1

### Non-Oxidative Dehydrogenation of Methanol to Methyl Formate (MF) Through Highly Stable and Reusable Cu/MgO Catalysts

Non-oxidative dehydrogenation of methanol to methyl formate over a CuMgO-based catalyst was investigated. Although the active site was metallic copper) (Cu⁰), the reaction conditions can be adjusted by tuning the ratio of Cu/Mg and optionally doping the catalyst with Pd to achieve a very specific activity for methyl formate synthesis. On the basis of CO₂-TPD study, the catalyst basic strength was a factor for efficient conversion of methanol to methyl formate via dehydrogenation. These CuMgO-based catalysts exhibited excellent thermal stability during the reaction and the regeneration processes. Approx. 80% methanol conversion with constant selectivity to methyl formate was achieved, even after 4 rounds of usage for a total reaction time exceeding 200 hours, indicative of the catalysts' potential for practical applications.

The direct dehydrogenation reaction of methanol to produce methyl formate, among other products, can be represented as shown in Scheme 1 below:

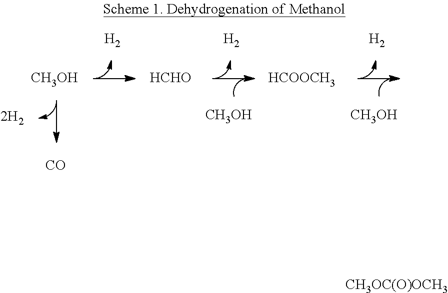

The present Example reports that the formation rate of the MF can be enhanced by the addition of a basic material (MgO) and the catalytic performance can be improved by tuning the ratio of Cu/MgO. Compared to conventional impregnated copper-based catalysts, the CuMgO-based catalysts exhibited a significant improvement of methanol conversion (11.7 to 16.7%) and MF selectivity (62.5 to 88.1%). Furthermore, the incorporation of Pd to CuMgO was found to prevent deactivation and increase the stability of the catalyst for methanol dehydrogenation.

### Experimental Section

### Catalyst Preparation

CuMgO catalysts were prepared by the co-precipitation method. Certain amounts (Cu/MgO weight ratio=about 1:9, 2:8, 3:7, 1:1, and 7:3) of Cu(NO₃)₂.3H₂O (Sigma-Aldrich, ≥99%) and Mg(NO₃)₂.6H₂O (Sigma-Aldrich, ≥99%) were dissolved in about 100 mL of D.I. water, and a 0.6 M K₂CO₃ (Sigma-Aldrich, ≥99%) solution was then added dropwise under vigorous stirring at about 70° C. for about 3 h. The blue solid co-precipitates were filtered and washed with D.I. water and then dried overnight at about 90° C. and finally calcined at about 400° C. for about 3 h (these catalysts were labeled as CuₓMgO_{y} where the x/y is the weight ratio of Cu/MgO).

Pd-containing catalysts were prepared by the incipient wetness impregnation method. CuMgO catalysts were impregnated with a given amount of Pd(NO₃)₂.2H₂O (Aldrich, ˜40% Pd basis) in an aqueous solution at about 60° C., and stirred for about 3 h. After impregnation, the samples were dried at about 90° C. overnight and then calcined at about 300° C. for about 4 h (these catalysts are labeled as xPd/CuMgO, with x representing the weight % of Pd in CuMgO).

### Characterization

CO₂-TPD and H₂-TPR were performed using the apparatus ALTAMIRA AMI 200 Ip (Altamira Instruments, Inc.) and detected by a Hiden HPR 20 mass spectrometer (Hiden Analytical, Inc.), 30 mg of each catalyst was used for measurement. CO₂-TPD samples were pre-treated for about 120 min at about 250° C. under argon (about 50 mL/min) then treated in a flux of about 1% of CO₂ in helium (about 25 mL/min) for about 60 min at room temperature, followed by helium flush for about 1 h at about 50° C. The temperature of the calorimeter furnace was then programmed with a heating rate of about 10° C./min, at a about 50 mL/min of helium flow rate. Samples of H2-TPR were pre-treated for about 120 min at about 250° C. under argon (50 mL/min), then cooled to about room temperature for about 60 min. Once the catalysts were stabilized, they underwent a reduction process in the calorimeter furnace a heating rate of about 10° C./min, and at about 5% H₂ in Ar at about 30 mL/min.

Samples were imaged using a Titan CT (FEI Company) operating at 300 kV and equipped with a 4 k×4 k CCD camera (Gatan Inc., Pleasanton, Calif.). They were placed on a 300 mesh copper grid precoated with a holey amorphous carbon film.

X-ray diffraction (XRD) spectra of the catalysts were obtained using a D8 Advance XRD (Bruker) at 40 kV and 40 mA, with CuKa (1.54184 Å) as the X-ray source. Diffraction patterns were recorded between 10° and 70° (2θ), by incremental steps of 0.02, at 10 deg/min.

Texture properties (e.g. specific surface areas, pore size, pore volume) of the catalysts were determined using the single-point BET (Brunauer, Emmett, Teller) method, by adsorption of N₂ at its liquid temperature and subsequent desorption at room temperature. ASAP 2420 apparatus (Micromeritics) was used in the experiments. Samples were degassed at 673 K for 10 hours, prior to analysis.

XPS experiments were performed using a Kratos Axis Ultra DLD instrument equipped with a monochromatic Al Kα X-ray source (hv=1486.6 eV) operated at a power of 150 W, and under UHV conditions in the range of ˜10-9 mbar. All spectra were recorded in hybrid mode using electrostatic and magnetic lenses and an aperture slot of 300 μm×700 μm. The survey and high-resolution spectra were acquired at fixed analyzer pass energies of 160 eV and 20 eV, respectively. The samples were mounted in floating mode in order to avoid differential charging. Therefore, XPS spectra were acquired using charge neutralization.

The TG analysis was performed using a Mettler Toledo TGA/DSC (thermal gravimetric analysis/differential scanning calorimetry) instrument equipped with a GC 200 Gas Controller and an auto-sampler. The sample was first subjected to an isothermal treatment of 150° C. for 30 minutes, and under flowing N₂ (99.9999% purity) with a flow rate of 50 ml/min. [Please confirm time unit, which was omitted in draft manuscript] It was subsequently heated to 1000° C. (10° C./min).

### Catalytic Reaction

The methanol dehydrogenation reaction was carried out in a fixed-bed reactor. Prior to the reaction, about 100 mg of catalyst was placed in the reactor and reduced by dilute hydrogen (H₂: N₂=about 30 mL/min: about 20 mL/min) for about 3 h. After pretreatment, a stream of pure methanol solution was first fed (about 0.1 mL/min) into an evaporator at about 200° C., then the methanol vapor was carried into the reactor by N₂ (about 50 mL/min). Reaction products were analyzed using an online GC system (Varian GC-450) connected with two channels, A and B. Channel A is consisted of a set of three packed columns, “Hayesep” Q (CP81073), “Hayesep” T (CP81072), and “Molsieve” 13X (CP81073) connected with a TCD detector to monitor CO and CO₂. Channel B uses a CP-wax 52CB column (CP7668) and was connected with a FID detector to monitor MF and other oxygenates. After a reaction time of about 50 hours, the spent catalyst was regenerated by calcination for about 3 h at about 400° C., under air atmosphere, to remove the coking. Once regenerated, the catalyst was reduced by dilute H₂ before undergoing the next round of catalytic test as previously described.

### Results and Discussion

### Characterization of Catalysts

Texture properties (Table 1) of the catalysts demonstrated that they all have similar BET surface areas (about 50.1-62.4 m²/g) and pore volumes (about 0.54-0.60 cm³/g), except the highest copper-containing catalyst (Cu₇MgO₃—CP), which had a larger pore size. The N₂ adsorption-desorption isotherm plot shows that the adsorption amount increased slowly with increasing pressure during the initial stage, and after the pressure reached the saturated vapor pressure, the adsorption amount increased rapidly. These pores were all provided by the crystal stacking (type III, non-porous material). These results led to the conclusion that Cu₇MgO₃—CP produced the largest particles, resulting in a larger pore size. These large particles could be a result of a copper aggregation, triggered by the higher copper loading. After the addition of palladium, the BET surface area and pore volume both decreased, due to the presence of the palladium heteroatom, as shown in 1Pd/Cu₃MgO₇ and 1Pd/Cu₅MgO₅.

TABLE 1
Textural properties of catalysts

| | | | | Particle size of Cu |
|---|---|---|---|---|
| Catalyst | S_{BET}(m²/g) | Dₚ(nm) | Vₚ(cm³/g) | (nm)^{a} |
| Cu₁MgO₉-CP | 50.1 | 132.1 | 0.54 | — |
| Cu₂MgO₈-CP | 32.7 | 128.1 | 0.20 | — |
| Cu₃MgO₇-CP | 49.6 | 129.4 | 0.75 | 9.46 |
| 1Pd/Cu₃MgO₇ | 47.5 | 122.9 | 0.23 | 8.84 |
| Cu₅MgO₅-CP | 67.4 | 152.6 | 0.68 | 11.88 |
| 1Pd/Cu₅MgO₅ | 20.5 | 151.7 | 0.38 | 11.02 |
| Cu₇MgO₃-CP | 62.4 | 330.0 | 0.60 | 23.63 |

^{a}Calculated by Scherrer equation.

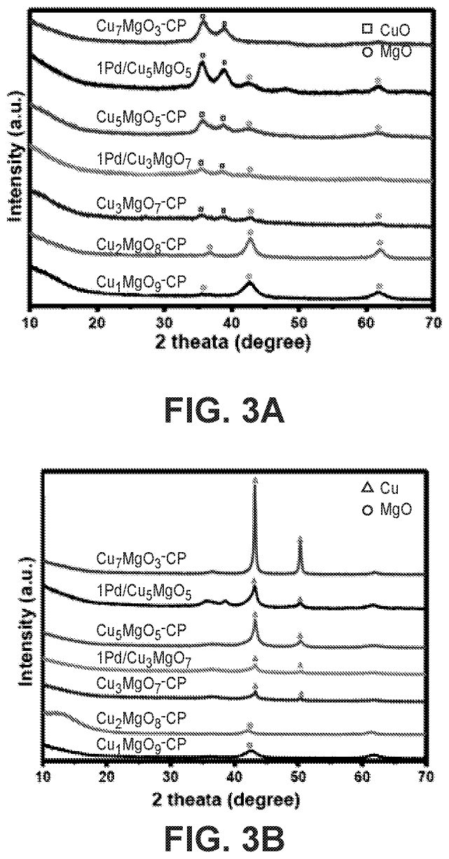

To better understand the effects of Pd and the Cu/MgO ratio, several techniques were employed to characterize the properties of the catalysts. In FIG. 3A, the wide-angle XRD patterns show that the intensity of CuO peaks (35.6°, 38.8°, JCPDS card no. 01-073-6023) increased with increasing copper loading, whereas the peaks of MgO (36.8°, 42.7°, 61.9°, JCPDS card no. 1-071-1176) decreased. The spent catalysts all showed Cu (43.2°, 50.3°, JCPDS cards no. 00-004-0836) and MgO peaks, but without those of CuO or Cu₂O (FIG. 3B). These results indicated that the metallic state of copper species (Cu⁰) could be maintained after the dehydrogenation reaction and the catalysts were not oxidized during the catalytic test. The particle sizes of metallic copper increased with increasing copper loading due to the aggregation of copper species. Moreover, the addition of Pd likely inhibited the growth of copper crystal, as the Pd-containing catalyst showed smaller particle sizes than those of catalysts without Pd.

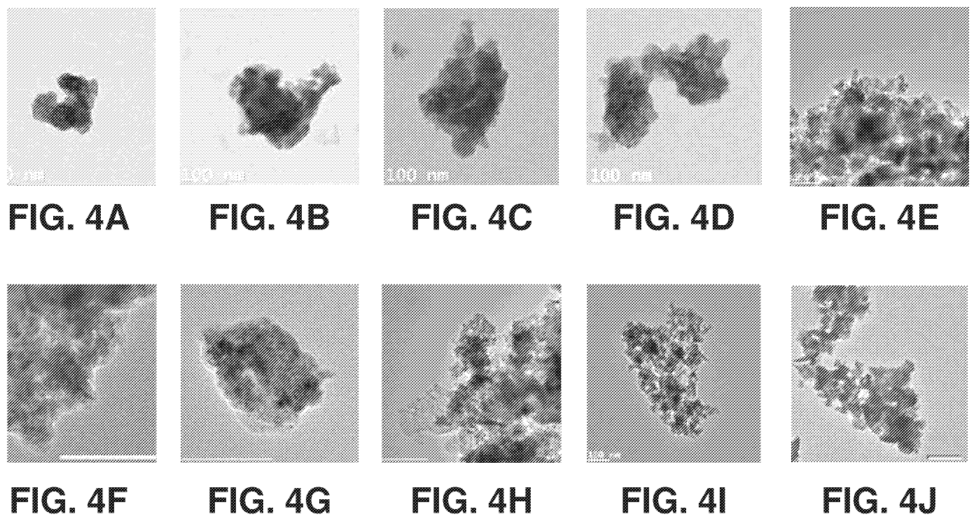

TEM images (FIGS. 4A-4J) show the significant differences in morphology between the high copper-containing and low copper-containing catalysts. The low copper-containing catalysts (FIGS. 4A, 4C, 4E, and 4G) show a mixture of copper nanocluster and MgO homogeneously with only small copper particles distributed on the MgO flakes. For the high copper-containing catalyst (FIG. 4I), the small copper nanoparticles became larger copper clusters, consistent with the results from the XRD analysis. Moreover, the study of spent catalysts revealed that the catalysts with palladium (FIGS. 4D, 4G) showed less amorphous carbon coking layer compared to those without palladium doping (FIGS. 4B, 4F) after the catalytic test. These observations suggested that the addition of palladium may inhibit the coke formation.

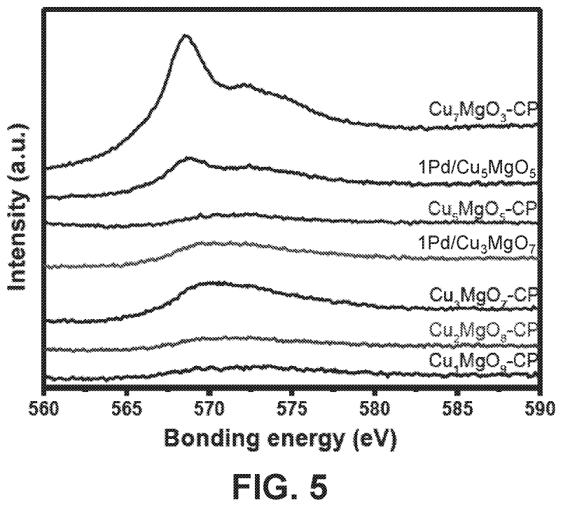

Although XRD patterns and TEM images provided useful information about the catalysts' surfaces, the XPS measurement offered more details about the higher copper-containing catalysts, particularly Cu₇MgO₃—CP. The Cu-LMM spectrum (FIG. 5) shows a shoulder at approx. 572.2 eV, indicating that the Cu₇MgO₃—CP catalyst had a more complex structure with larger copper clusters compared to others, further confirming the results in the XRD and TEM studies.

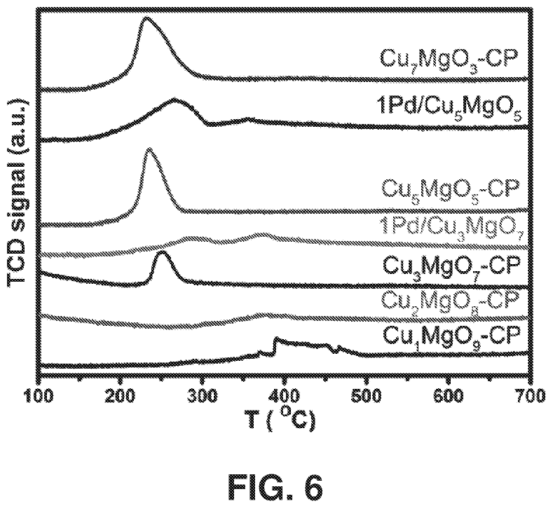

Additionally, the H₂-TPR measurement (FIG. 6) shows that the low copper-containing catalysts (Cu₁MgO₉—CP and Cu₂MgO₈—CP) possessed almost no ability to be reduced. The amount of H₂ consumption increased with the increasing copper loading, but the reduction temperature almost stayed the same (about 250° C.); these findings suggested that the preparation processes using the co-precipitation method successfully enhanced the amount of surface copper, and all the copper-based catalysts had similar reduction sites with different amounts of H₂ consumption. The H₂-TPR measurement also revealed the effects of the addition of palladium. The reduction peak slightly shifted to higher temperatures to approx. 350° C., presumably due to the interaction of the small palladium particles with hydrogen, which enhanced the reduction ability of the copper oxide through the dissociation of H₂ on palladium followed by the spillover on the copper oxide phase.

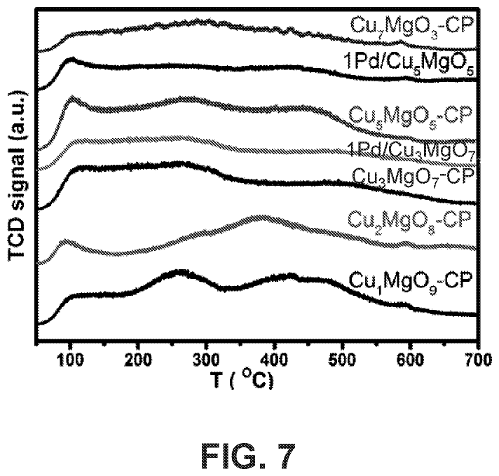

In the CO₂-TPD study, the strength of basic sites were found to increase with the magnesium content (FIG. 7). The high copper-containing (low magnesium) catalysts only showed a weak basic site (100-150° C.), and the Cu₇MgO₃—CP catalyst showed almost no basic sites. Both Cu₁MgO₉—CP and Cu₂MgO₈—CP, however, afforded medium-strong basic sites. The addition of palladium heteroatom reduced the amount of medium-strong basic sites as well as the total amount of basic sites, and these findings were directly supported by the activity of the catalysts in the methanol dehydrogenation reaction.

### Activity Test

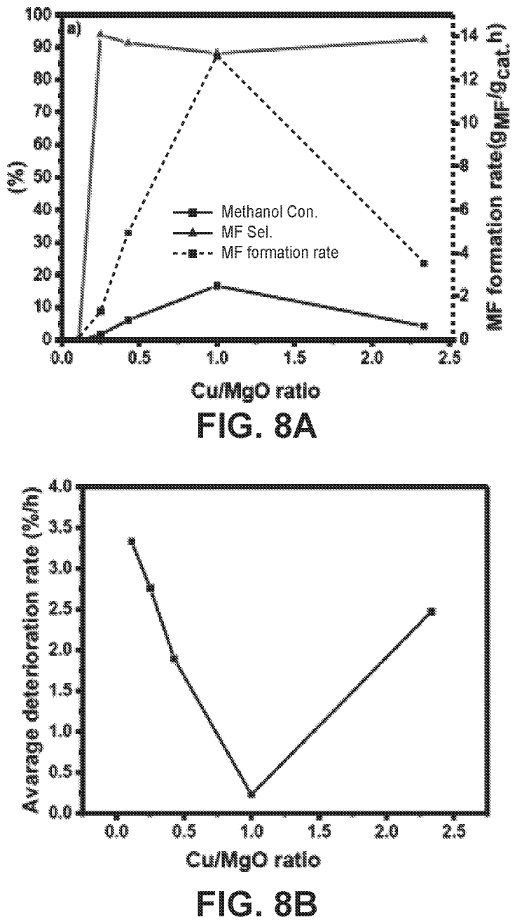

In the catalyst screening, the dramatic influence of the Cu/MgO ratio on the catalytic performance was observed. Table 2 and FIG. 8A show that a specific ratio of Cu/MgO was necessary for a methanol dehydrogenation. Although a higher copper ratio improved the conversion, the excess amount of copper harmed the catalytic performance, presumably due to the fact that the high copper concentration can lead to bigger copper clusters, whereas lower copper loading favored the formation of smaller copper nanoparticles. Higher copper-containing catalysts, such as Cu₅MgO₅ and Cu₇MgO₃, also displayed a higher CO selectivity compared to those of the low copper-containing catalysts. The conversion decreased as the reaction time increased, and this deactivation was likely related to the coke formation. As the copper loading increased, the lifetime became longer, but only up to the Cu/MgO ratio of 1. The catalyst lifetime then decreased for those of Cu/MgO ratios higher than 1 (FIG. 8B). Again, these observations suggested that the larger copper clusters were formed in the high copper loading rather than copper nanoparticles, thus offering fewer active sites. In addition to the copper effect, an insufficient MgO content provided almost no basic sites (CO₂-TPD, FIG. 7), and resulted in the decrease of the catalytic performance Excess MgO, which offered more medium-strong basic sites, also harmed the catalytic performance, because the stronger basic sites likely facilitated other side reactions such as polymerizations and formation of polycyclic aromatics. These side reactions can form more coke and deactivate the catalyst faster.

TABLE 2
Catalyst Screen

| | | | | | | | Carbon | Coking |
|---|---|---|---|---|---|---|---|---|
| | | Rate | Conversion | Selectivity (%) | | | Balance | wt. |
| Entry | Catalyst | (g_{MF}/g_{cat} · h) | (%) | MF | CO | CO₂ | (%) | (%) |
| 1 | Cu₁MgO₉-CP | — | — | — | — | — | — | 22 |
| 2 | Cu₂MgO₈-CP | 1.3 | 1.6 | 93.8 | 1.2 | 2.4 | 97.4 | 26.5 |
| 3 | Cu₃MgO₇-CP | 4.9 | 6.1 | 91.3 | 2.4 | 2.5 | 96.2 | 30.6 |
| 4 | 1Pd/Cu₃MgO₇ | 12.4 | 15.0 | 93.1 | 1.6 | 1.6 | 96.3 | 19.1 |
| 5 | Cu₅MgO₅-CP | 13.1 | 16.7 | 88.1 | 5.5 | 2.4 | 96.0 | 15.9 |
| 6 | 1Pd/Cu₅MgO₅ | 12.4 | 14.9 | 93.3 | 4.1 | <0.5 | 97.4 | 11.0 |
| 7 | Cu₇MgO₃-CP | 3.6 | 4.3 | 92.3 | 5.2 | <0.5 | 97.5 | 15.6 |

Reaction condition: T = 250° C., carrier gas flow (N₂) = 50 mL/min, feedstock (liquid methanol) = 0.1 mL/min, catalyst = 100 mg, WHSV_{meOH} = 47.4 h⁻¹, 30 h.

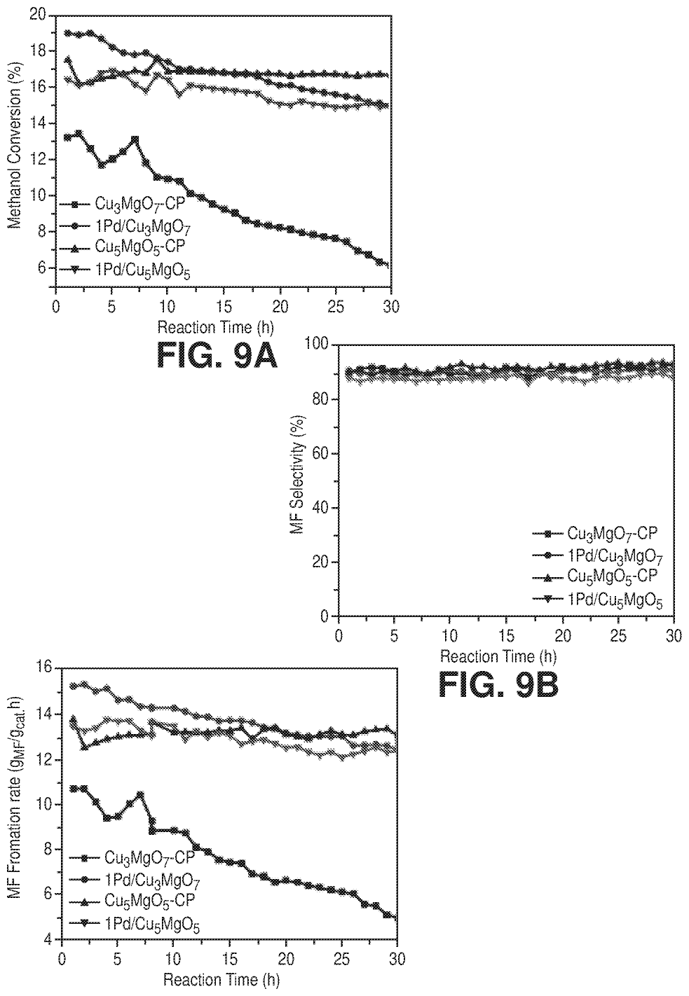

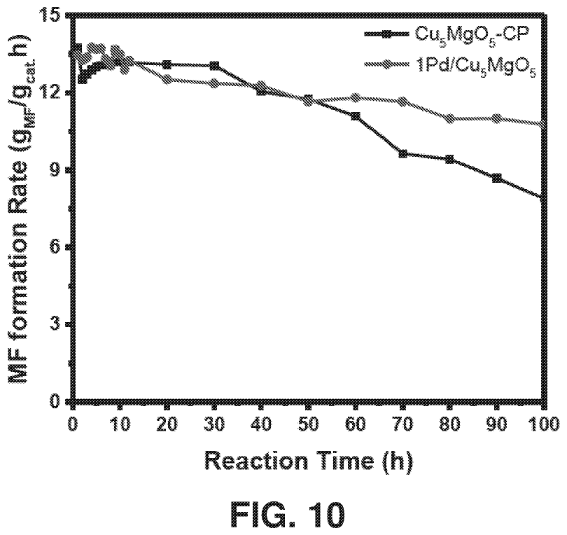

The addition of palladium can improve the performance and stability of the catalyst because palladium inhibited the medium-strong basic sites (FIG. 7) and extended the lifetime of the catalyst. The excess of medium-strong basic sites in high MgO-containing catalysts promoted more side reactions to form the coke to deactivate the catalyst. Moreover, the addition of palladium also decreased the reduction ability of the catalyst to lower the selectivity towards gaseous products (such as CO and CO₂) and to enhance MF selectivity (Table 2, entries 3-6). As shown in FIGS. 9A-9C, the low copper-containing catalysts (the Cu₃MgO₇—CP and 1Pd/Cu₃MgO₇—CP) showed significant differences in the catalytic activity (methanol conversion=6.1 and 15.0%), and Cu₅MgO₅—CP and 1Pd/Cu₅MgO₅ catalysts provided a similar methanol conversion (16.7 and 14.9%) and MF selectivity (88.1 and 93.3%). The long-term test showed that 1Pd/Cu₅MgO₅ could maintain about 80% of the original activity over the course of reaction time of about 100 hours (FIG. 10, 79.2% of methanol conversion and 80.0% MF formation rate), while the activity of the Cu₅MgO₅ catalyst was dropped by approx. 40% (56.9% of methanol conversion and 57.5% of MF formation rate).

### Reuse Test

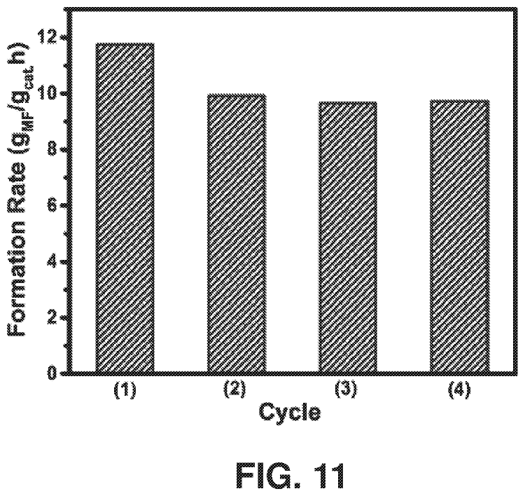

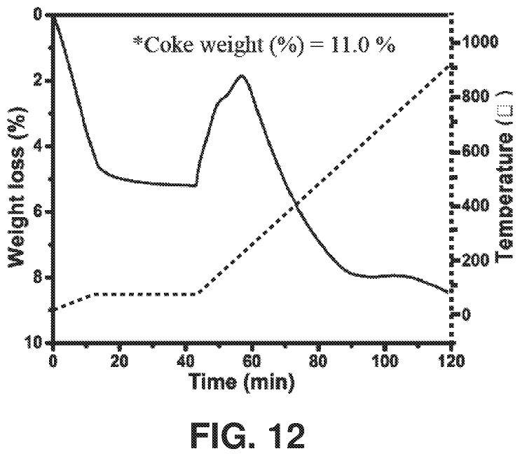

Results of the reuse test on the Cu₅MgO₅ catalyst indicated that after 4-round usage, the catalyst still maintained about 80.1% of activity in the MF formation rate (FIG. 11). The activity test after the second round led to the similar results (MF formation rate=9.9, 9.6, and 9.7 g_{MF}/g_{cat}·h); however, the performance was slightly lower than those obtained for the first round, suggesting that a few active sites could be poisoned after the first round and could not be regenerated effectively. The weight loss of the spent catalyst (FIG. 12) suggested that some of the coke could not be removed under about 600° C. and this unremovable hard coke might have caused the deactivation of the regenerated catalysts.

In sum, this Example revealed that an optimized value of the Cu/MgO ratio facilitated the methanol dehydrogenation reaction. The excessive copper loading formed larger copper clusters, rather than copper nanoparticles, which lowered the MF selectivity. On the other hand, the excess MgO led to the increase of coke formation due to the higher medium-strong basic sites. These two effects both harmed the catalytic performance (i.e. methanol conversion and lifetime of the catalyst). The Cu/MgO ratio of 1 provided the best catalytic performance. In addition, Pd also played a critical role to prolong the lifetime of the catalyst by decreasing the amount of medium-strong basic sites. The addition of Pd not only modified the catalyst's basic property with a smaller amount of medium-strong basic sites, but also enhanced the reduction ability of copper oxide to improve the catalytic activity and stability. The reuse test demonstrated that the Cu₅MgO₅—CP catalyst can be efficiently regenerated and reused for at least 4 rounds. After the regeneration process (through calcination), at least 80% methanol conversion and constant MF selectivity can be achieved, suggesting a great potential for practical applications in a low-cost process for the MF production.

Other embodiments of the present disclosure are possible. Although the description above contains much specificity, these should not be construed as limiting the scope of the disclosure, but as merely providing illustrations of some of the presently preferred embodiments of this disclosure. It is also contemplated that various combinations or sub-combinations of the specific features and aspects of the embodiments may be made and still fall within the scope of this disclosure. It should be understood that various features and aspects of the disclosed embodiments can be combined with or substituted for one another in order to form various embodiments. Thus, it is intended that the scope of at least some of the present disclosure should not be limited by the particular disclosed embodiments described above.

Thus the scope of this disclosure should be determined by the appended claims and their legal equivalents. Therefore, it will be appreciated that the scope of the present disclosure fully encompasses other embodiments which may become obvious to those skilled in the art, and that the scope of the present disclosure is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims.

The foregoing description of various preferred embodiments of the disclosure have been presented for purposes of illustration and description. It is not intended to be exhaustive or to limit the disclosure to the precise embodiments, and obviously many modifications and variations are possible in light of the above teaching. The example embodiments, as described above, were chosen and described in order to best explain the principles of the disclosure and its practical application to thereby enable others skilled in the art to best utilize the disclosure in various embodiments and with various modifications as are suited to the particular use contemplated. It is intended that the scope of the disclosure be defined by the claims appended hereto

Various examples have been described. These and other examples are within the scope of the following claims.

1. A catalyst composition for non-oxidative dehydrogenation of methanol to methyl formate, the catalyst composition comprising:
catalytic nanoparticles distributed on a surface of a promoter, wherein the catalytic nanoparticles include Cu nanoparticles and the promoter includes MgO.

2. The catalyst composition of claim 1, wherein the catalyst composition is a pre-catalyst including one or more of CuO nanoparticles and Cu₂O nanoparticles.

3. The catalyst composition of claim 1, wherein the catalyst composition is a catalyst including Cu⁰ nanoparticles.

4. The catalyst composition of claim 1, wherein the catalyst composition is represented by formula (I):
CuₓMgO_{y} (I)
wherein x/y is a weight ratio of Cu/MgO.

5. The catalyst composition of claim 4, wherein the catalyst composition is Cu₁MgO₉, Cu₂MgO₀, Cu₃MgO₇, Cu₅MgO₅, or Cu₇MgO₃.

6. The catalyst composition of claim 1, further composing Pd.

7. The catalyst composition of claim 6, wherein the catalyst composition is represented by formula (II):
zPd/CuₓMgO_{y}
wherein z is a wt % of Pd and x/y is a weight ratio of Cu/MgO.

8. The catalyst composition of claim 7, wherein the catalyst composition is 1Pd/Cu₃MgO₇ or 1Pd/Cu₅MgO₅.

9. A method of preparing a pre-catalyst, comprising:
dissolving a Cu precursor and a Mg precursor in water to form a precursor solution;
adding a precipitating agent to the precursor solution to form a co-precipitate including Cu and Mg; and
calcining the co-precipitate at a first select temperature to form a pre-catalyst.

10. The method of claim 9, wherein the Cu precursor is Cu(NO₃)₂.3H₂O, CuSO₄.3H₂O, CuCl₂, CuBr₂, or Cu(OAc)₂.3H₂O; wherein the Mg precursor is Mg(NO₃)₂.6H₂O; wherein the precipitating agent is K₂CO₃, NH₄OH, NaOH, (NH₄)₂CO₃, or Na₂CO₃.

11. The method of claim 9, wherein the pre-catalyst includes one or more of CuO, Cu₂O, and MgO.

12. The method of claim 9, further comprising impregnating the pre-catalyst with a Pd precursor to obtain an impregnated pre-catalyst and calcinating the impregnated pre-catalyst at a second select temperature.

13. The method of claim 9, further comprising reducing the pre-catalyst in H₂ at a third select temperature to obtain a catalyst including metallic Cu nanoparticles distributed on a surface of MgO.

14. A method of producing at least methyl formate by non-oxidative dehydrogenation of methanol, comprising:
flowing a fluid composition containing at least methanol over a catalyst to produce methyl formate and hydrogen;
wherein the catalyst is the catalyst composition of claim 1 and wherein the catalyst includes Cu nanoparticles distributed on a surface of MgO; and
recovering one or more of the methyl formate and hydrogen.

15. The method of claim 14, further comprising reducing a pre-catalyst in H₂ at a select temperature to obtain the catalyst.

16. The method of claim 15, wherein the pre-catalyst includes one or more of CuO, Cu₂O, and MgO.

17. The method of claim 14, wherein the Cu nanoparticles are metallic Cu nanoparticles.

18. The method of claim 14, wherein the catalyst composition achieves a methanol conversion of about 14% or greater.

19. The method of claim 14, wherein the catalyst composition exhibits a methyl formate selectivity of about 80% or greater.

20. The method of claim 14, wherein the catalyst composition achieves a methyl formate formation rate of about 10 g_{MF}/g_{cat}·h or greater.
